(19) United States
(12) Patent Application Publication
(10) Pub. No.: US 2026/0123997 A1
(43) Pub. Date: May 7, 2026

(54) MARKER POSITIONING METHOD, DEVICE AND SYSTEM BASED ON MODEL FUSION

(71) Applicant: HUNAN ZHUOSHI CHUANGSI TECHNOLOGY CO., LTD., Changsha (CN)

(72) Inventor: Dan TANG, Changsha (CN)

(21) Appl. No.: 19/439,425

(22) Filed: Jan. 4, 2026

Related U.S. Application Data
(63) Continuation of application No. PCT/CN2024/104088, filed on Jul. 5, 2024.

(30) Foreign Application Priority Data
Jul. 6, 2023 (CN) 202310825931.4

(51) Int. Cl. A61B 34/10; A61B 90/00; G06T 7/73; G06T 17/00; G06T 19/00

(52) U.S. Cl. CPC A61B 34/10; A61B 90/39; G06T 7/75; G06T 17/00; G06T 19/00; A61B 2034/107; A61B 2090/3916; A61B 2090/3954; A61B 2090/3966; A61B 2090/3995; G06T 2207/10081; G06T 2207/10088; G06T 2207/30096; G06T 2207/30204; G06T 2210/41

(57) ABSTRACT

Provided are a marker positioning method, device and system based on model fusion, which are applicable to the technical field of intelligent manufacturing. The method includes the following steps: acquiring pre-scanning data corresponding to a target site of a patient; constructing a structural model corresponding to the target site of the patient by using the pre-scanning data, the structural model containing surface features and internal features of the target site; acquiring surface scanning data corresponding to the target site of the patient; constructing a surface contour model on the basis of the surface scanning data; and fusing the structural model and the surface contour model to obtain a surgical guidance model, the surgical guidance model indicating position parameters of a marker.

### CROSS-REFERENCE TO RELATED APPLICATIONS

This application is a continuation of International Patent Application No. PCT/CN 2024/104088 with a filing date of Jul. 5, 2024, designating the United States, now pending, and further claims priority to Chinese Patent Application No. 202310825931.4 with a filing date of Jul. 6, 2023. The content of the aforementioned applications, including any intervening amendments thereto, are incorporated herein by reference.

### TECHNICAL FIELD

The embodiments in the specification relate to the technical field of intelligent manufacturing, particularly to a marker positioning method, device and system based on model fusion.

### BACKGROUND OF THE PRESENT INVENTION

It is very important to determine a position of a deep structure of a patient when performing a surgery. A tomographic image of a scanned site can be acquired by CT or MR scanning, and then a relative position relationship between an internal deep structure and a surface marker structure can be acquired by a three-dimensional reconstruction technology, so that the position of the deep structure is positioned on the basis of a position of a surface recognizable structure during the surgery, for example, a position of an internal hematoma is positioned through a position of a surface marker, such as an external auditory canal and an eye, so as to plan a surgical approach.

However, in practical application, due to the lack of obvious recognizable structures in some surgical sites, such as the absence of the marker structure on a surface of brain, the low resolving accuracy of the recognizable structure on a surface of the site, and other factors, the positioning performed by directly using the surface recognizable structure in some application scenarios is poor in effect and accuracy. Aiming at this problem, an artificial marker, such as an electrode patch, a fiducial marker, a skull pin and a stereotactic frame, may be set on the surface of the surgical site of the patient, and then the artificial marker is identified and positioned.

At present, when the marker is used, the marker is usually attached to a specific position of the surgical site of the patient first, then the patient is subjected to CT scanning, and finally, CT scanning results acquired include overall scanning results of the surgical site and position information of the marker at the same time. However, this method has a high requirement of coordination between a marker device and radiology scanning, which cannot even be completed in an emergency surgery. In addition, this method needs to ensure that the position of the marker must be fixed from the setting of the marker to the execution of surgery, but a non-invasive marker is difficult to meet this requirement, while an invasive marker may bring an additional wound to the patient. At present, the method of positioning the depth structure on the basis of the marker does not have good application effect. Therefore, there is an urgent need for a convenient and effective positioning method based on the marker.

### SUMMARY OF THE PRESENT INVENTION

The embodiments in the specification aim to provide a marker positioning method, device and system based on model fusion, so as to solve the problem about how to position a marker conveniently and effectively.

In order to solve the above technical problem, the embodiments in the specification provide a marker positioning method based on model fusion, which includes the following steps: acquiring pre-scanning data corresponding to a target site of a patient, wherein the pre-scanning data are scanning data acquired before setting a marker on a surface of the target site of the patient; constructing a structural model corresponding to the target site of the patient by using the pre-scanning data, wherein the structural model contains surface features and internal features of the target site; acquiring surface scanning data corresponding to the target site of the patient, wherein the surface scanning data are scanning data corresponding to the surface of the target site of the patient after setting the marker on the surface of the target site of the patient; constructing a surface contour model on the basis of the surface scanning data; and fusing the structural model and the surface contour model to obtain a surgical guidance model, wherein the surgical guidance model indicates the position parameters of the marker.

In some embodiments, the pre-scanning data include CT/MR scanning data; and the surface scanning data include scanning data acquired by a three-dimensional scanner.

In some embodiments, the pre-scanning data include a tomographic image; and the step of constructing the structural model corresponding to the target site of the patient by using the pre-scanning data, includes: determining a surface contour structure according to a peripheral shape of the tomographic image; determining an internal tissue structure according to inner features of the tomographic image; and constructing the structural model corresponding to the target site of the patient according to the surface contour structure and the internal tissue structure.

In some embodiments, the step of constructing the surface contour model on the basis of the surface scanning data, includes: constructing a candidate scanning model according to the surface scanning data; and identifying and removing a background region from the candidate scanning model to obtain the surface contour model.

In some embodiments, the step of fusing the structural model and the surface contour model to obtain the surgical guidance model, includes: determining outer contour features of the surface contour model; and fusing the structural model and the surface contour model to obtain the surgical guidance model on the basis of the outer contour features of the surface contour model and the surface features of the structural model.

In some embodiments, after the step of fusing the structural model and the surface contour model to obtain the surgical guidance model, the method further includes the following steps: determining position parameters of a lesion according to internal features in the surgical guidance model; and constructing a surgical approach by using the position parameters of the lesion and the position parameters of the marker, wherein the surgical approach has a pose relationship corresponding to position parameters of each marker.

In some embodiments, after the step of fusing the structural model and the surface contour model to obtain the surgical guidance model, the method further includes the following steps: determining a model registration accuracy according to the surgical guidance model; and when the model registration accuracy meets a model fusion requirement, applying the surgical guidance model to intelligent manufacturing, or when the model registration accuracy does not meet the model fusion requirement, adjusting the surgical guidance model.

In some embodiments, the marker includes at least one of an electrode patch, a fiducial marker, a skull pin and a skin marker.

The embodiments in the specification further provide a marker positioning device based on model fusion, which includes: a pre-scanning data acquiring module, configured for acquiring pre-scanning data corresponding to a target site of a patient, wherein the pre-scanning data are scanning data acquired before setting a marker on a surface of the target site of the patient; a structural model constructing module, configured for constructing a structural model corresponding to the target site of the patient by using the pre-scanning data, wherein the structural model contains surface features and internal features of the target site; a surface scanning data acquiring module, configured for acquiring surface scanning data corresponding to the target site of the patient, wherein the surface scanning data are scanning data corresponding to the surface of the target site of the patient after setting the marker on the surface of the target site of the patient; a surface contour model constructing module, configured for constructing a surface contour model on the basis of the surface scanning data; and a model fusing module, configured for fusing the structural model and the surface contour model to obtain a surgical guidance model, wherein the surgical guidance model indicates the position parameters of the marker.

The embodiments in the specification further provide a marker positioning system based on model fusion, which includes a pre-scanning device, a three-dimensional scanning device and a computing device; wherein, the pre-scanning device is used for scanning a target site of a patient to obtain pre-scanning data, wherein the pre-scanning data are scanning data acquired before setting a marker on a surface of the target site of the patient; and the pre-scanning data include data obtained by scanning surface and internal parts; the three-dimensional scanning device is used for scanning the target site of the patient to obtain surface scanning data, wherein the surface scanning data are scanning data corresponding to the surface of the target site of the patient after setting the marker on the surface of the target site of the patient; and the computing device stores a computer program instruction, and the computing device is used for acquiring the pre-scanning data and the surface scanning data, and executing the computer program/instruction to implement the steps of the marker positioning method based on model fusion above.

The embodiments in the specification further provide a computer storage medium, on which a computer program/instruction is stored, wherein, the computer program instruction, when executed, is used for implementing the steps of the marker positioning method based on model fusion above.

It can be seen from the technical solutions provided by the embodiments in the specification that, according to the marker positioning method, device and system based on model fusion above, the pre-scanning data are acquired before setting the marker on the surface part of the patient, and the surface scanning data are acquired after setting the marker. The structural model is constructed according to the pre-scanning data, the surface contour model is constructed according to the surface scanning data, and finally, the surgical guidance model is obtained by fusing the structural model and the surface contour model. The scanning and the model construction are performed before setting the marker, without considering a setting requirement of the marker; and only the surface scanning data are acquired after setting the marker, which can not only speed up the scanning, but also ensure the stability of the position of the marker. Finally, the surgical guidance model is obtained through model fusion, which can not only determine a spatial pose of an internal tissue, but also determine the position of the surface marker, so that the marker and the lesion can be effectively positioned by the surgical guidance model, and then a surgical procedure is effectively guided, thereby ensuring surgical accuracy and operability.

### DESCRIPTION OF THE DRAWINGS

In order to illustrate the technical solutions in the embodiments in the specification and in the prior art more clearly, the drawings to be used in the description of the embodiments or the prior art will be briefly described below. Obviously, the drawings in the following description are merely some embodiments in the specification. Those of ordinary skills in the art can also obtain other drawings based on these drawings without going through any creative work.

FIG. 1 is a structural diagram of a marker positioning system based on model fusion according to the embodiments in the specification;

FIG. 2 is a flow chart of a marker positioning method based on model fusion according to the embodiments in the specification;

FIG. 3 is a flow chart of an executing process of a positioning surgery based on model fusion according to the embodiments in the specification; and

FIG. 4 is a diagram of modules of a marker positioning device based on model fusion according to the embodiments in the specification.

### DETAILED DESCRIPTION OF PREFERRED EMBODIMENTS

Technical solutions in the embodiments in the specification will be clearly and completely described hereinafter with reference to the drawings in the embodiments in the specification. Obviously, the described embodiments are only some but not all of the embodiments in the specification. Based on the embodiments in the specification, all other embodiments obtained by those of ordinary skills in the art without going through any creative work shall fall within the scope of protection of the specification.

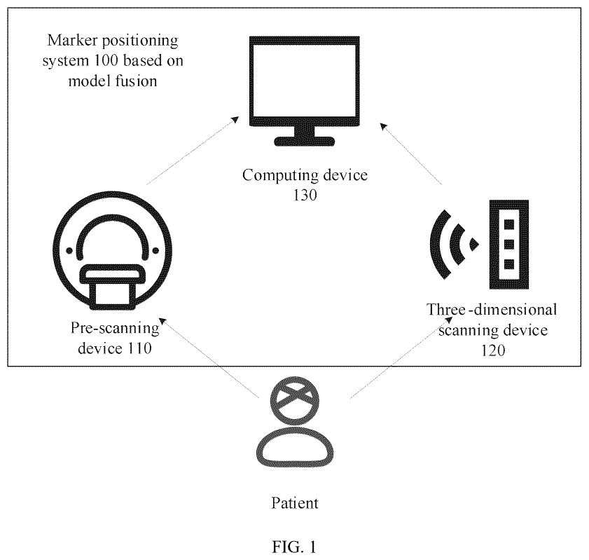

In order to better understand the inventive concept of the present application, a marker positioning system based on model fusion according to the embodiments in the specification is introduced first. As shown in FIG. 1, the marker positioning system 100 based on model fusion includes a pre-scanning device 110, a three-dimensional scanning device 120 and a computing device 130.

The pre-scanning device 110 may be used to scan a patient, so as to acquire pre-scanning data of a specific site. For example, when the pre-scanning device 110 is used to scan a skull of the patient, an external shape of the skull of the patient can be determined, and a distribution status of an intracerebral tissue and a position of a lesion in the skull can also be determined.

Specifically, the pre-scanning device 110 may be, for example, a CT scanning device, an MR scanning device or a nuclear magnetic resonance device, and scanning data acquired may be CT/MR scanning data or nuclear magnetic resonance detection data. In practical application, other devices with a function of scanning a deep site of the patient are taken as the pre-scanning device 110, which will not be limited.

Because of a powerful scanning function of the pre-scanning device 110, in practical application, the pre-scanning device 110 is also large in volume, and often fixed in a radiology room. On the basis of a marker setting method in the prior art, a marker needs to be set first, and then the patient is scanned together with the marker. When a model is constructed by using scanning data, the model contains position parameters of the marker at the same time, so that a surgical plan is made. However, because the marker is used for positioning during a surgery, the surgery needs to be performed within a short time after setting the marker, which requires a high degree of time coordination between a radiology department and an operating room, and there is a certain risk of falling off of the marker. The above situation makes it very inconvenient to directly apply the pre-scanning device 110 to acquire the scanning data corresponding to the marker and the patient.

The three-dimensional scanning device 120 is used for acquiring an outer contour structure of an object. For example, the three-dimensional scanning device 120 may acquire corresponding point cloud data under corresponding scenarios, and a distance from each position on a surface of the object to the scanning device can be determined through the point cloud data, so that the outer contour of the object can be determined according to the point cloud data. Because the three-dimensional scanning device 120 is only used to acquire the outer contour, the three-dimensional scanning device 120 may have a small volume, for example, the three-dimensional scanning device may be a handheld device, which means that the three-dimensional scanning device 120 may be applied in the operating room to directly scan the patient before a surgery.

Specifically, the three-dimensional scanning device 120 may be a three-dimensional scanner, which can directly acquire three-dimensional scanning data of the outer contour of the object. In practical application, other devices may also be applied as the three-dimensional scanning device 120 as required, which will not be limited.

The computing device 130 may communicate with the pre-scanning device 110 and the three-dimensional scanning device 120, receive scanning data produced by the pre-scanning device 110 and the three-dimensional scanning device 120, and processing the scanning data acquired, so as to perform model construction, model fusion, and other operations.

Specifically, the computing device 130 may include a memory and a processor. In this embodiment, the memory may be achieved in any suitable way. For example, the memory may be a read-only memory, a hard disk drive, a solid-state drive, a USB flash drive, or the like. The memory may be used for storing a computer program/instruction, so as to perform scanning data processing, model construction, model fusion, and other operations.

In this embodiment, the processor may be achieved in any suitable way. For example, the processor may take the form of, for example, a microprocessor or a processor and a computer-readable medium storing a computer-readable program code (such as software or firmware) executable by the (micro) processor, a logic gate, a switch, an application specific integrated circuit (ASIC), a programmable logic controller and an embedded microcontroller, and the like. The processor may execute the computer program instruction to implement the following steps: acquiring pre-scanning data corresponding to a target site of a patient, wherein the pre-scanning data are scanning data acquired before setting a marker on a surface of the target site of the patient; constructing a structural model corresponding to the target site of the patient by using the pre-scanning data, wherein the structural model contains surface features and internal features of the target site; acquiring surface scanning data corresponding to the target site of the patient, wherein the surface scanning data are scanning data corresponding to the surface of the target site of the patient after setting the marker on the surface of the target site of the patient; constructing a surface contour model on the basis of the surface scanning data; and fusing the structural model and the surface contour model to obtain a surgical guidance model, wherein the surgical guidance model indicates the position parameters of the marker. Specific introduction of the executed content will be described in detail in steps of a subsequent execution method, which will not be repeated herein.

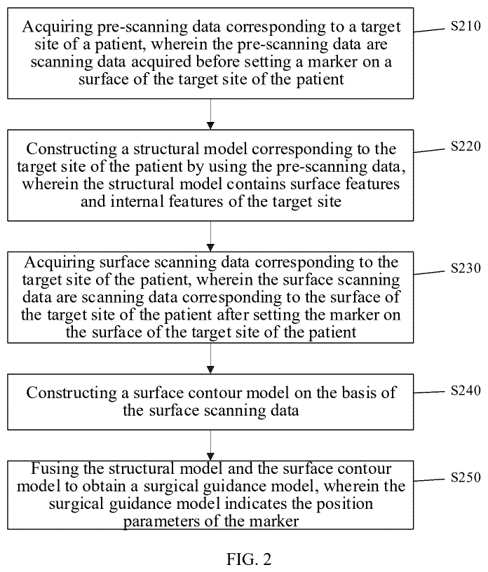

On the basis of the marker positioning system based on model fusion, the embodiments in the specification provide a marker positioning method based on model fusion. An executing subject of the marker positioning method based on model fusion may be the computing device in the marker positioning system based on model fusion. As shown in FIG. 2, the marker positioning method based on model fusion includes the following specific implementation steps.

In S210, pre-scanning data corresponding to a target site of a patient are acquired, wherein the pre-scanning data are scanning data acquired before setting a marker on a surface of the target site of the patient.

The pre-scanning data are scanning data corresponding to the target site of the patient acquired by the pre-scanning device above. In the embodiments in the specification, the pre-scanning data are scanning data acquired before setting a marker on a surface of the target site of the patient. When there is no marker on the surface part of the patient, there is no need to fix the marker, which means that the pre-scanning data may be acquired by the pre-scanning device at any time before a surgery.

The target site of the patient is a site on which the marker needs to be set for positioning during the surgery. For example, when performing a surgery on a skull, because the skull is smooth and lacks an obvious identification point, it is necessary to set the identification point on a surface of the skull for positioning. In practical application, other sites on which the markers need to be set may also be determined as the target sites, which will not be limited.

The pre-scanning data contain not only scanning data corresponding to an outer contour of the target site, but also scanning data corresponding to a deep tissue structure of the target site. Different tissues may be distinguished through the scanning data, and accordingly, when a model is constructed according to the scanning data in subsequent step, a distribution status of an internal tissue of the model can be reflected at the same time.

In some embodiments, the pre-scanning data may also may a tomographic image, the tomographic image may be a tomographic image obtained by scanning the target site at a specific interval, and a model corresponding to the target site can also be constructed on the basis of the tomographic image.

In some examples, the pre-scanning data may also be DICOM data. The DICOM data may be medical digital imaging and communication data, corresponding to X-ray, CT, nuclear magnetic resonance, ultrasound, and other radiation diagnosis and treatment means, which can effectively reflect a status of a scanned site through a scanning image.

In S220, a structural model corresponding to the target site of the patient is constructed by using the pre-scanning data, wherein the structural model contains surface features and internal features of the target site.

According to the pre-scanning data acquired, the corresponding structural model may be constructed. The structural model can reflect an outer contour of the target site, and a distribution status of the internal tissue and a distribution status of the lesion. According to the structural model, a corresponding surgical approach can be planned, for example, when it is necessary to puncture to a position of the lesion, a corresponding puncture path may be planned without damaging the internal tissues, so that a puncture point of the puncture path on the surface of the target site is determined to guide a corresponding surgical operation.

Therefore, the structural model may contain the surface features and the internal features of the target site. The surface features may include the outer contour of the target site or a corresponding surface feature point. For example, when the target site is the skull, the surface features include an external shape of the skull, and shapes and positions of an eye, a nose, an external auditory canal, and the like on the skull. The internal features may include distribution statuses of internal tissue, organ and the like of the target site, or a position and a distribution range of an internal lesion. For example, when the target site is the skull, the internal features may include a spatial distribution status of a brain tissue and a position of a lesion on the brain tissue.

In general, the pre-scanning data reflect features of a corresponding site, and corresponding tissues or organs of the site may be deduced according to the displayed features, so as to complete the construction of the structural model.

In some embodiments, when the pre-scanning data are the tomographic image, because different tomographic images correspond to sectional scanning results in different positions, and a distance between different tomographic images is fixed, a peripheral shape and inner features of the target site may be distinguished in the tomographic image first. The inner features may include sectional shapes and positions of various organs and tissues, then the surface contour structure is fitted according to the peripheral shape of each tomographic image and the internal tissue structure is fitted according to the inner features, and the structural model of the target site of the patient may be completed according to the surface contour structure and the internal tissue structure.

In some embodiments, when the pre-scanning data are CT/MR scanning data, the CT/MR scanning data completed before the surgery may also be processed into imaging source models (dicom data models) of an outer contour model of head and face skin and an internal interest structure model by a DICOM data processing module, and the structure model can also be constructed. Specifically, when the pre-scanning data are DICOM data, the DICOM data may be read first to perform surface contour segmentation and internal structure segmentation in sequence, and finally, a corresponding dicom source model is produced.

In practical application, other ways of producing the structural model according to the pre-scanning data may be set according to requirements, which are not limited to the above examples, and will not be repeated herein.

It should be noted that there is no restriction on an execution order of this step in practical application, and the structural model may be constructed before setting the marker on the surface part of the patient, or the structural model may be constructed after setting the marker on the surface part of the patient, which may be determined according to an actual application requirement.

In S230, surface scanning data corresponding to the target site of the patient are acquired, wherein the surface scanning data are scanning data corresponding to the surface of the target site of the patient after setting the marker on the surface of the target site of the patient.

The surface scanning data corresponding to the target site of the patient may be acquired after setting the marker on the surface of the target site of the patient. The surface scanning data only relate to scanning of the surface part of the patient, without involving scanning of a deep structure. According to the introduction about the three-dimensional scanning device above, because of convenient and easy-to-use features of the three-dimensional scanning device, the three-dimensional scanning device may be used to scan the patient quickly before starting the surgery, or directly arranged in the operating room to scan the patient directly before performing the surgery. Because of this feature of the three-dimensional scanning device, the marker may be set on the surface part of the patient before starting the surgery, and the surgery may be performed after completing the scanning quickly.

In practical application, the step S210 may be executed at any time to acquire the pre-scanning data of the patient. Because the surface and internal parts of the patient will not change obviously in a short time, the structure of the target site corresponding to the pre-scanning data can remain stable for a long time. The step S230 may be implemented before performing the surgery, which means that, in the case of positioning with the marker during the surgery, the position of the marker is set first, and then the surface part of the patient is scanned together with the marker.

In a specific application scenario, the marker may be a device with certain geometric features, wherein the geometric features may be three-dimensional features, or two-dimensional features capable of being identified by the scanning device. The marker may be, for example, an electrode patch, a fiducial marker, a skull pin and a skin marker. In practical application, other devices capable of being effectively fixed on the surface part of the patient and capable of being scanned by the three-dimensional scanning device may be taken as the markers, which will not be limited.

In S240, a surface contour model is constructed on the basis of the surface scanning data.

Because the surface scanning data can reflect the surface features such as the contour and the shape of the target site, the surface contour model may be constructed according to the surface scanning data. The surface contour model is used for reflecting the surface features of the target site and the position parameters of the marker.

A specific construction process, for example, may refer to constructing the corresponding surface contour model according to a distance reflected by point cloud data in the case that the surface scanning data are the point cloud data by the way as described above, or refer to obtaining multi-angle images of the target site, and then constructing the corresponding surface contour model according to the features of the target site in these images. In practical application, the corresponding way may be chosen as required to complete the construction of the surface contour model, which will not be repeated herein.

When the three-dimensional scanning device is used to scan the target site, it is inevitable to scan background objects together. In order to ensure that the finally constructed model corresponds to the outer contour of the target site, in some embodiments, a candidate scanning model may be constructed first according to the surface scanning data. The candidate scanning model is a model constructed according to all the surface scanning data, which may include not only the target site of the patient, but also models corresponding to the background objects, such as a pillow and a platform.

A background region may be identified and removed from the candidate scanning model to obtain the surface contour model. The removal process may be implemented manually by an operator, or features of the background region may be acquired in advance, and the background region may be removed by comparing the background region with the candidate scanning model, for example, the background region is simply scanned in advance, and then the background region is removed by comparison and the surface contour model is constructed.

In S250, the structural model and the surface contour model are fused to obtain a surgical guidance model, wherein the surgical guidance model indicates the position parameters of the marker.

After acquiring the structural model and the surface contour model, the two models may be fused to obtain the surgical guidance model. Because the structural model contains the relevant internal features of the target site, and the surface contour model contains a setting status of the marker on the surface of the target site, the surgical guidance model obtained after fusion can not only reflect a surface contour and a distribution status of the marker, but also reflect a distribution status of an internal tissue, thereby providing effective guidance for a surgical procedure.

Specifically, the surgical guidance model indicates the position parameters of the marker, the position parameters of the marker are used to indicate the position of the marker on a surface of the surgical guidance model, the marker can be effectively positioned through the position parameters of the marker, and a specific point can be positioned on the surface part of the patient on the basis of a position of the specific point relative to the marker.

Because each of the structural model and the surface contour model contains the outer contour of the target site, and in general, the outer contours in the two models are the same, the model fusion can be achieved by matching the outer contours. Specifically, outer contour features of the surface contour model may be determined, wherein the outer contour features may include an external shape of the model, a position and a shape of a surface feature point, and the like. On the basis the outer contour features of the surface contour model and the surface features of the structural model, a matching relationship between the models is determined, so that the features can correspond to each other, thereby completing the model fusion.

In practical application, the models may also be fused by other ways or algorithms, which are not limited to the above examples, and will not be repeated herein.

Accordingly, a surgical approach may be determined according to the surgical guidance model. A lesion region may be determined according to the internal features of the surgical guidance model, thereby determining position parameters of a lesion of the lesion region. On the basis of the position parameters of the lesion and the distribution status of the internal tissue, the surgical approach for the target site may be planned according to a surgical requirement. When the surgical approach is projected on the surgical guidance model, there is a spatial position relationship with respect to each marker at the same time, which means that the surgical approach has a pose relationship corresponding to position parameters of each marker. The surgical execution approach can be effectively determined on the surface part of the patient according to the position of the marker on the basis of this pose relationship, for example, when the surgical approach is a puncture path, the puncture path corresponds to a puncture point on the surface part of the patient, and a position of the puncture point may be determined according to the position of the marker, and a puncture direction can also be determined on the basis of position relationships with respect to a plurality of markers, so that a puncture operation can be effectively implemented.

In some embodiments, after completing the construction of the structural model, because the structural model can already reflect the distribution statuses of the internal tissue and the lesion, a surgical plan may be set directly according to the structural model, including the determination of the surgical approach based on the structural model. After completing the model fusion to produce the surgical guidance model, the surgical approach may be directly transplanted to the surgical guidance model according to a position relationship of the surgical approach on the structural model, and a position relationship of the surgical approach with respect to the marker is determined, which can also achieve a surgical guidance effect.

In some embodiments, after completing the model fusion, a registration accuracy may also be evaluated for the surgical guidance model. Specifically, the model registration accuracy may be determined according to the surgical guidance model first, and the model registration accuracy may be a parameter obtained by comparing the surgical guidance model with the structural model/surface contour model to determine a difference between the models and quantifying the difference.

The model registration accuracy is compared with a preset model fusion requirement, wherein the model fusion requirement may be a preset condition for limiting a model fusion effect. If the model registration accuracy meets the model fusion requirement, the surgical guidance model may be applied to complete a corresponding surgical guidance operation during the surgery; and if the model registration accuracy does not meet the model fusion requirement, the surgical guidance model may be adjusted, wherein the adjustment may refer to performing the model fusion again on a differential site, or performing manual adjustment according to the differential site. The specific way to adjust the surgical guidance model may be set according to a requirement of practical application, which will not be limited.

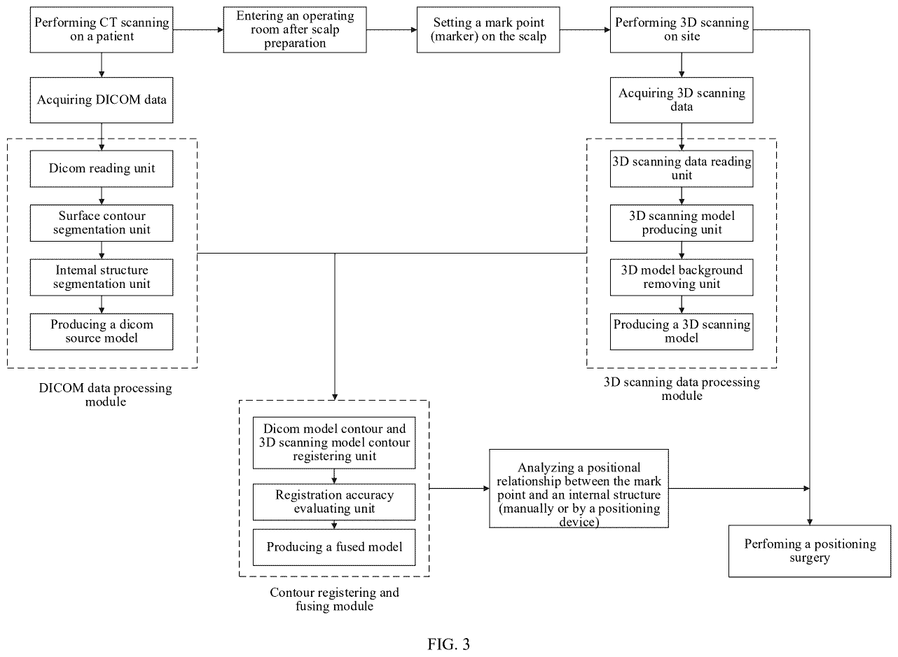

With reference to the scenario example in FIG. 3, the flow of the marker positioning method based on model fusion above is exemplarily introduced. As shown in FIG. 3, the patient is subjected to the CT scanning first, and the DICOM data are acquired. The dicom source model is produced through a dicom reading unit, a surface contour segmentation unit and an internal structure segmentation unit in the DICOM data processing module, which is the structural model. Meanwhile, in another flow, the patient enters the operating room after scalp preparation (head shaving), a mark point is set on the scalp, and 3D scanning is performed on the spot to obtain 3D scanning data. A 3D scanning model is produced for the 3D scanning data through a 3D scanning data reading unit, a 3D scanning model producing unit and a 3D model background removing unit in the 3D scanning data processing module, which is the surface contour model.

The dicom source model and the 3D scanning model are jointly input into a contour registering and fusing module, and registered through a dicom model contour and 3D scanning model contour registering unit, and the registration accuracy is evaluated through a registration accuracy evaluating unit, so as to finally produce a fused model, which is the surgical guidance model. A positional relationship between the mark point and the internal structure can be analyzed through model fusion, and finally, the positioning surgery can be effectively performed.

It can be seen from the embodiment and the introduction of the scenario example above that, according to the marker positioning method and system based on model fusion, the pre-scanning data are acquired before setting the marker on the surface part of the patient, and the surface scanning data are acquired after setting the marker. The structural model is constructed according to the pre-scanning data, the surface contour model is constructed according to the surface scanning data, and finally, the surgical guidance model is obtained by fusing the structural model and the surface contour model. The scanning and the model construction are performed before setting the marker, without considering a setting requirement of the marker; and only the surface scanning data are acquired after setting the marker, which can not only speed up the scanning, but also ensure the stability of the position of the marker. Finally, the surgical guidance model is obtained through model fusion, which can not only determine a spatial pose of an internal tissue, but also determine the position of the surface marker, so that the marker and the lesion can be effectively positioned by the surgical guidance model, and then a surgical procedure is effectively guided, thereby ensuring surgical accuracy and operability.

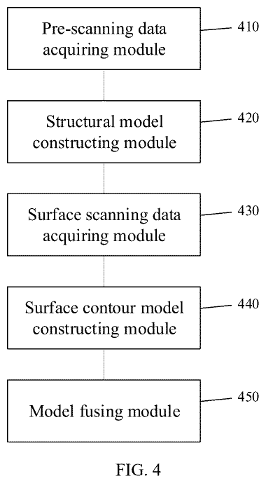

On the basis of the marker positioning method based on model fusion as shown in FIG. 2, the embodiments in the specification further provide a marker positioning device based on model fusion. The marker positioning device based on model fusion may be arranged on the computing device. As shown in FIG. 4, the marker positioning device based on model fusion includes the following specific modules.

A pre-scanning data acquiring module 410 is configured for acquiring pre-scanning data corresponding to a target site of a patient, wherein the pre-scanning data are scanning data acquired before setting a marker on a surface of the target site of the patient.

A structural model constructing module 420 is configured for constructing a structural model corresponding to the target site of the patient by using the pre-scanning data, wherein the structural model contains surface features and internal features of the target site.

A surface scanning data acquiring module 430 is configured for acquiring surface scanning data corresponding to the target site of the patient, wherein the surface scanning data are scanning data corresponding to the surface of the target site of the patient after setting the marker on the surface of the target site of the patient.

A surface contour model constructing module 440 is configured for constructing a surface contour model on the basis of the surface scanning data.

A model fusing module 450 is configured for fusing the structural model and the surface contour model to obtain a surgical guidance model, wherein the surgical guidance model indicates the position parameters of the marker.

On the basis of the marker positioning method based on model fusion as shown in FIG. 2, the embodiments in the specification provide a computer-readable storage medium on which a computer program/instruction is stored. The computer-readable storage medium may be read by a processor on the basis of an internal bus of a computing device, so that the program instruction in the computer-readable storage medium is executed by the processor.

In this embodiment, the computer-readable storage medium may be achieved in any suitable way. The computer-readable storage medium includes, but is not limited to, a Random Access Memory (RAM), a Read-Only Memory (ROM), a Cache, a Hard Disk Drive (HDD), a Memory Card, and the like. The computer storage medium stores the computer program instruction. When the computer program instruction is executed, the program/instruction or the module in the embodiment corresponding to FIG. 2 in the specification is implemented.

It should be noted that the marker positioning method, device and system based on model fusion above are applicable to the technical field of intelligent manufacturing, or are applicable to other technical fields, which will not be limited.

Although the process flow described above includes a plurality of operations in a specific order, it should be clearly understood that these processes may include more or fewer operations, which may be performed sequentially or in parallel (such as using a parallel processor or a multi-threaded environment).

The present application is described with reference to the flow charts and/or block diagrams of the method, the device (system), and the computer program product according to the embodiments in the specification. It should be appreciated that each flow and/or block in the flow charts and/or block diagrams, and combinations of the flows and/or blocks in the flow charts and/or block diagrams may be implemented by computer program instructions. These computer program instructions may be provided to a general purpose computer, a special purpose computer, an embedded processor, or a processor of other programmable data processing apparatus to produce a machine for the instructions executed by the computer or the processor of other programmable data processing apparatus to generate a device for implementing the functions specified in one or more flows of the flow chart and/or in one or more blocks of the block diagram.

These computer program instructions may also be provided to a computer readable memory that can guide the computer or other programmable data processing apparatus to work in a given manner, so that the instructions stored in the computer readable memory generate a product including an instruction device that implements the functions specified in one or more flows of the flow chart and/or in one or more blocks of the block diagram.

These computer program instructions may also be loaded to a computer, or other programmable data processing apparatus, so that a series of operating steps are executed on the computer, or other programmable data processing apparatus to produce processing implemented by the computer, so that the instructions executed in the computer or other programmable data processing apparatus provide steps for implementing the functions specified in one or more flows of the flow chart and/or in one or more blocks of the block diagram.

In a typical configuration, the computing device includes one or more processors (CPU), input/output interfaces, a network interface and a memory.

The memory may include forms such as a non-permanent memory, a random access memory (RAM) and/or a nonvolatile memory in the computer-readable medium, such as a read-only memory (ROM) or a flash memory. The memory is an example of the computer-readable medium.

The computer-readable medium, including permanent and non-permanent, and removable and non-removable media, may store information by any method or technology. The information may be a computer-readable instruction, a data structure, a program module, or other data. Examples of the computer storage medium include, but are not limited to, a phase change memory (PRAM), a static random access memory (SRAM), a dynamic random access memory (DRAM), other types of random access memories (RAM), a read-only memory (ROM), an electrically erasable programmable read-only memory (EEPROM), a flash memory or other memory technologies, a compact disc read-only memory (CD-ROM), a digital video disk (DVD) or other optical storages, a magnetic cassette tape, magnetic tape and magnetic disk storages or other magnetic storage devices, or any other non-transmission medium, which may be used for storing information accessible to a computing device. According to the definition herein, the computer-readable medium does not include transitory media, such as modulated data signals and carrier waves.

Those skilled in the art shall understand that the embodiments in the specification may be provided as the method, the system or the computer program product. Therefore, the embodiments in the specification may take the form of complete hardware embodiments, complete software embodiments or software-hardware combined embodiments. Moreover, the embodiments in the specification may take the form of computer program product implemented on one or more computer usable storage media (including, but being not limited to, a disk memory, a CD ROM, an optical memory, etc.) in which computer usable program codes are included.

The embodiments in the specification may be described in the general context of the computer-executable instruction executed by the computer, such as a program module. Generally, the program module includes a routine, a program, an object, an assembly, a data structure, and the like, which are used for executing a particular task or achieving a particular abstract data type. The embodiments in the specification may also be practiced in distributed computing environments, and in these distributed computing environments, a task is performed by a remote processing device connected through a communication network. In the distributed computing environments, the program module may be located in local and remote computer storage media including the storage device.

The embodiments in the specification are all described in a progressive way, only the same and similar parts between the embodiments may be referred to each other, and each embodiment focuses on the differences from other embodiments. Particularly, the system embodiments are basically similar to the method embodiments, so that the description is relatively simple, and the related part may refer to the part of the method embodiments. In the descriptions of the specification, the descriptions with reference to the terms “one embodiment”, “some embodiments”, “example”, “specific example” or “some examples”, etc., refer to that specific features, structures, materials or features described with reference to the embodiments or examples are included in at least one embodiment or example of the embodiments in the specification. In the specification, the schematic representation of the above terms does not necessarily refer to the same embodiment or example. Moreover, the specific features, structures, materials or features described may be combined in any one or more embodiments or examples in a suitable manner. In addition, in the case of having no mutual contradiction, those skilled in the art may join and combine different embodiments or examples described in the specification and the features of the different embodiments or examples.

The above are only the embodiments of the present application, and are not intended to limit the present application. For those skilled in the art, the present application may have various modifications and changes. Any modification, equivalent substitution, improvement, and the like made without departing from the spirit and principle of the present application should fall within the scope of the claims of the present application.

We claim:

1. A marker positioning method based on model fusion, comprising the following steps:
acquiring pre-scanning data corresponding to a target site of a patient, wherein the pre-scanning data are scanning data acquired before setting a marker on a surface of the target site of the patient;
and the marker comprises an artificial marker set on the surface;
constructing a structural model corresponding to the target site of the patient by using the pre-scanning data, wherein the structural model contains surface features and internal features of the target site;
acquiring surface scanning data corresponding to the target site of the patient, wherein the surface scanning data are scanning data corresponding to the surface of the target site of the patient after setting the marker on the surface of the target site of the patient;
constructing a surface contour model on the basis of the surface scanning data, wherein the surface contour model comprises the surface features and position parameters of the marker; and
fusing the structural model and the surface contour model to obtain a surgical guidance model, wherein the surgical guidance model indicates the position parameters of the marker.

2. The method according to claim 1, wherein the pre-scanning data comprise CT/MR scanning data; and the surface scanning data comprise scanning data acquired by a three-dimensional scanner.

3. The method according to claim 1, wherein the pre-scanning data comprise a tomographic image; and the step of constructing the structural model corresponding to the target site of the patient by using the pre-scanning data, comprises:
determining a surface contour structure according to a peripheral shape of the tomographic image;
determining an internal tissue structure according to inner features of the tomographic image; and
constructing the structural model corresponding to the target site of the patient according to the surface contour structure and the internal tissue structure.

4. The method according to claim 1, wherein the step of constructing the surface contour model on the basis of the surface scanning data, comprises:
constructing a candidate scanning model according to the surface scanning data; and
identifying and removing a background region from the candidate scanning model to obtain the surface contour model.

5. The method according to claim 1, wherein the step of fusing the structural model and the surface contour model to obtain the surgical guidance model, comprises:
determining outer contour features of the surface contour model; and
fusing the structural model and the surface contour model to obtain the surgical guidance model on the basis of the outer contour features of the surface contour model and the surface features of the structural model.

6. The method according to claim 1, wherein, after the step of fusing the structural model and the surface contour model to obtain the surgical guidance model, the method further comprises the following steps:
determining position parameters of a lesion according to internal features in the surgical guidance model; and
constructing a surgical approach by using the position parameters of the lesion, wherein the surgical approach has a pose relationship corresponding to position parameters of each marker.

7. The method according to claim 1, wherein, after the step of fusing the structural model and the surface contour model to obtain the surgical guidance model, the method further comprises the following steps:
determining a model registration accuracy according to the surgical guidance model; and
when the model registration accuracy meets a model fusion requirement, applying the surgical guidance model to a surgery, or when the model registration accuracy does not meet the model fusion requirement, adjusting the surgical guidance model.

8. A marker positioning device based on model fusion, comprising:
a pre-scanning data acquiring module, configured for acquiring pre-scanning data corresponding to a target site of a patient, wherein the pre-scanning data are scanning data acquired before setting a marker on a surface of the target site of the patient; and the marker comprises an artificial marker set on the surface;
a structural model constructing module, configured for constructing a structural model corresponding to the target site of the patient by using the pre-scanning data, wherein the structural model contains surface features and internal features of the target site;
a surface scanning data acquiring module, configured for acquiring surface scanning data corresponding to the target site of the patient, wherein the surface scanning data are scanning data corresponding to the surface of the target site of the patient after setting the marker on the surface of the target site of the patient;
a surface contour model constructing module, configured for constructing a surface contour model on the basis of the surface scanning data, wherein the surface contour model comprises the surface features and position parameters of the marker; and
a model fusing module, configured for fusing the structural model and the surface contour model to obtain a surgical guidance model, wherein the surgical guidance model indicates the position parameters of the marker.

9. A marker positioning system based on model fusion, comprising a pre-scanning device, a three-dimensional scanning device and a computing device; wherein,
the pre-scanning device is used for scanning a target site of a patient to obtain pre-scanning data, wherein the pre-scanning data are scanning data acquired before setting a marker on a surface of the target site of the patient; the pre-scanning data comprise data obtained by scanning surface and internal parts; and the marker comprises an artificial marker set on the surface;
the three-dimensional scanning device is used for scanning the target site of the patient to obtain surface scanning data, wherein the surface scanning data are scanning data corresponding to the surface of the target site of the patient after setting the marker on the surface of the target site of the patient; and
the computing device stores a computer program/instruction, and the computing device is used for acquiring the pre-scanning data and the surface scanning data, and executing the computer program/instruction to implement the steps of the method according to any one of claims 1 to 7.

10. A computer storage medium, on which a computer program/instruction is stored, wherein, the computer program/instruction, when executed, is used for implementing the steps of the method according to any one of claims 1 to 7.
